(19) United States
(12) Patent Application Publication
(10) Pub. No.: US 2022/0411845 A1
(43) Pub. Date: Dec. 29, 2022

(54) METHOD FOR ENUMERATION OF BACTERIA IN LIQUID SAMPLES, AND SAMPLE HOLDER USEFUL FOR THIS METHOD

(71) Applicant: Microbium d.o.o., Ljubljana-Dobrunje (SI)

(72) Inventors: Rok Poglajen, Izlake (SI); Gregor Zupin, Ljubljana (SI); Sabina Ott Rutar, Pivka (SI)

(73) Assignee: Microbium d.o.o., Ljubljana-Dobrunje (SI)

(21) Appl. No.: 17/773,997

(22) PCT Filed: Nov. 3, 2020
(86) PCT No.: PCT/EP2020/080746

§ 371 Date: May 3, 2022

(30) Foreign Application Priority Data
Nov. 4, 2019 (SI) P-201900207

(51) Int. Cl. C12Q 1/06; C12M 1/00; C12M 1/34; C12Q 1/10

(52) U.S. Cl. CPC C12Q 1/06; C12M 23/34; C12M 41/36; C12Q 1/10; G01N 2333/21; G01N 2333/245; G01N 2333/32

(57) ABSTRACT

Disclosed is a method for detection and/or quantification of microorganism in a liquid sample, in particular in a water sample, the method comprising the steps of: (a) distributing the liquid sample into a number of different discrete volume portions in a linear distribution pattern, or diluting the liquid sample into a number of dilution samples by a dilution factor of a linear distribution pattern; (b) allowing the microorganism to grow; and (c) applying the Most Probable Number method to the linearly distributed volume portions or the linearly diluted dilution samples to detect and/or quantify the microorganism. The invention also discloses a sample holder for detection and/or quantification of microorganism in a liquid sample, wherein the sample holder is structured to hold the liquid sample in a number of different compartments, wherein the different compartments respectively define a linear volume distribution.

### TECHNICAL FIELD OF THE INVENTION

The present invention relates to a method for detection and/or quantification of microorganisms such as bacteria in liquid samples, preferably water samples, and also relates to a sample holder which is particularly useful for this method.

### BACKGROUND ART

Detection and quantification of microorganisms, such as bacteria, is a crucial painpoint of a range of different industrial fields: the drinking water sector, the wastewater sector, the bathing water sector, chemical industry (production of paints, pigments, colorants, slurry), food industry, composts and the pharmaceutical industry for at least two main reasons forming two opposite counter-forces. On one hand, all of these industrial sectors are facing microbial contaminations, which are compromising the quality of their products and simultaneously affecting the health of the users. On the other hand, they are subjected to strong regulatory pressures. In the Drinking water sector, for example, the Drinking Water Directive (98/83/EC) reqiures no E. coli, no enterococci and no coliform bacteria present in the water sample. For bottled water, also no P. aeruginsa should be present in the bottled water sample.

A well-known method for detection and estimation/quantification of microorganisms in a liquid sample is the Most Probable Number method (MPN method). The method is based on a random distribution of a total unknown number of bacterial cells dispersed in an initial defined Euclidian volume of the liquid sample into discrete compartments of smaller Euclidian volumes. Until now, two different series of statistical events (statistical operations) were used as standard in the MPN method. The first one is that a series of either 2x or 10x sample dilutions are made. All dilutions of the sample are then distributed in multiple repetitions in wells (spaces) of the same volume. The second one is that the sample containing a defined number (or density of bacterial cells) is distributed between different volumes with either 10x or 2x volume ratio with multiple repetitions of the same volume. Thus there are multiple possibilities of the composition of the compartments—either all compartments may be of the same volume or the compartments may follow a volume pattern of different volumes with either 2× or 10× volume ratio. Each different volume may be present in a multitude of repetitions. Both cases underlie a stochastic geometry process in which the initial unknown number of bacterial cells is or is not diluted beforehand and further randomly partitioned or distributed between several compartments of defined smaller Euclidian volumes, which may be of particular constitution regarding the number of different volumes and the number of repetitions of the same volume—for example the well of a microwell plate or a model modification thereof—and ultimately resides in a novel random spatial distribution in a novel abstract Euclidian space. This statistical process is composed of two steps: Firstly all possible outcomes of the mentioned partitioning an unknown initial number of cells (or dilutions thereof) partitioned in a novel deifned compartment of a particular Euclidian volume can be explained by the Poisson distribution. The equation for the Poisson probability function of the bacterial sample (representing the probability that j bacterial cells will be in a well of a defined volume) can be described as:

$$P = \frac{{( d V μ )}^{j} {e}^{- dV μ}}{j !}$$

where j represents the number of bacterial cells in the well, d is the dilution factor, V is the volume of the well, μ is the initial bacterial concentration (the most probable number of bacteria per mL of sample) and the product dVμ defines the number of bacterial cells. Each bacterial cell is a point independent of other points in the defined Euclidian mathematical place. Detecting at least a single bacterium in the novel well (or defined compartment) or the novel Euclidian stochastic space is interpreted as a positive result thus indicating the presence of microorganisms in the compartment in question. The absence of bacteria in a respective compartment is calculated from the Poisson distribution as:

$${p}_{absence} = \frac{{( d V μ )}^{j} {e}^{- dV μ}}{j !} = \frac{{( d V μ )}^{0} {e}^{- dV μ}}{0 !} = {e}^{- dV μ}$$

The probability of a presence of at least a single bacterium in a compartment with a respective volume is therefore the opposite of the probability of the absence of bacterial cells in the compartment in question and it is calculated as:

p_{presence}=1−e^{-dVμ}

The sample with an initial unknown number of bacterial cells is however distributed between multiplicities of compartments of different defined Euclidian volumes (following generally a specific volume pattern of either 10× or 2× volume ratio) with each different volume present in a multitude of repetitions. The second step in the statistical process is thus to calculate the probability of detecting at least a single bacterium (a positive result) in a multitude—a defined number—of wells of the same volume and/or dilution (repeptitions of the same volume and/or dilution) out of all copartments of the same volume and/or dilution and over all different volumes. The probability of detecting a positive result (the presence of at least a single bacterium) in multiple repetitions (from one to all repetitions) of the same defined dilution and/or volume can be further explained by the Binomial distribution. Calculating the probability of detecting at least a single bacterium a respective well of defined Euclidian volume (from the Poisson distribution) thus gives us the weighted probability of the further Binomial distribution and enables us to select a particular Binomial distribution out of a family of Binomial distributions. Let us for example describe a system with x different volumes and y repetitions of the same volume. The Binomial distribution would describe the probability of r positive compartments out of y compartments with the same volume and/or dilution and p_{presence} would describe the weighted probability calculated by the above referred Poisson distribution describing the probability of at least a single bacterial cell present in a compartment with a defined volume. P_{absence} would describe the weighted probability of absence of bacterial cells in a compartment of a particular volume:

$${P}_{b i n o m i a l} = \frac{y !}{r ! ( y - r ) !} {p}_{presence}^{r} x {p}_{absence}^{y - r} = \frac{y !}{r ! ( y - r ) !} {( 1 - {e}^{- dV μ} )}^{r} {( {e}^{- dV μ} )}^{y - r}$$

The system however is composed of x different volumes, with y repetitions of the same volume. The outcome or the final result is defined by the number of positives (rᵢ) out of yᵢ repetitinos of the same volume i over all different volumes (from i=1 to i=x). The final outcome of the MPN method is described by the likelihood function (L) and is calculated as the product of the Binomial distribution probabilities of obtaining the number of positive compartments (rᵢ) (compartments with at least a single bacterial cell present) out of yᵢ repetitinos of the same volume i for each of the different volumes (from i=1 to i=x). From the likelihood function, the natural logarithm of the likelihood function may be calculated. For the above described system with x different volumes and y repetitions of the same volume, the first derivative of the natural logarithm of this likelihood function with respect to μ can be described as:

$$\frac{d ln L}{d μ} = {\sum }_{i = 1}^{i = x} \frac{{r}_{i} {d}_{i} {V}_{i} {e}^{- {d}_{i} {V}_{i} μ}}{1 - {e}^{- {d}_{i} {V}_{i} μ}} - ( {y}_{i} - {r}_{i} ) {d}_{i} {V}_{i} = {\sum }_{i = 1}^{i = x} \frac{{r}_{i} {d}_{i} {V}_{i}}{1 - {e}^{- {d}_{i} {V}_{i} μ}} - {y}_{i} {d}_{i} {V}_{i}$$

Where r is the defined number of positive wells in repetitions of the same dilution and/or volume. The most probable bacterial concentration based on a defined outcome (a certain number of positives (rᵢ) out of yᵢ repetitinos of the same volume i over all different volumes (from i=1 to i=x)) would be the μ at which the natural logarithm of the likelihood function is maximal, which is when its first derivative with respect to μ is zero. The derived μ from the equation is the estimate of the most probable number of microorganisms per mL of sample. If the same initial bacterial concentration would be analysed an infinite number of times, the estimate of the most probable number of bacterial cells per mL of sample would predictively follow a normal distribution with a population average μₚ—indicating the exact (actual) number of bacterial cells and a population standard deviation σ_{μp}. Taking a sample of measurements from the infinite population of measurements, the sample of measurements predictively follows a t-student distribution with a sample average μₛ and a sample standard deviation s_{μp}. From each sample average and sample standard deviation, an interval can be calculated indicating the range of μ values (between the upper and the lower limit) inside which there is a 95% probability that the actual number of bacterial cells in the sample resides—the 95% confidence intervals. Also taking an infinite numbers of sample measurements from the population of sample measurements; each sample measurement defining its own 1) sample average μₛ, 2) sample standard deviation s_{μs} and 3) 95% confidence interval, 95% of the 95% confidence intervals would contain the population average μₚ, thus the exact or actual number of bacterial cells. After calculating an estimate of the Most Probable Number from equating the first derivative of the natural logarithm likelihood function with respect to μ, the variance of the estimate of the MPN can be calculated as the second derivative of the natural logarithm of the Likelihood function with respect to μ. From the variance, the estimate of the standard deviation of the MPN estimate can be calculated and the estimate of the standard deviation of the natural logarithm of the MPN estimate can be calculated by dividing the estimate of the standard deviation of the MPN estimate with the MPN estimate. From the estimate of the standard deviation of the natural logarithm of the MPN estimate, the 95% confidence intervals are calculated as:

I_{lower limit}=μe^{-2s}^{_{ln μ}}

I_{upper limit}=μe^{2s}^{_{ln μ}}

Where s_{ln μ} is the estimate of the standard deviation of the natural logarithm of the MPN estimate μ I_{lower limit} is the lower limit of the 95% confidence interval and the I_{upper limit} is the upper limit of the 95% confidence interval.

Each previously described industrial sector is either interested in detecting total bacteria present or is targeting the presence of a defined bacterial species, such as Escherichia coli, or a defined bacterial group, such as coliform bacteria. Obtaining positive results for a targeted bacterial group can be achieved by selective differential chromogenic or fluorogenic media. The selective diferential chromogenic components are conjugates of substrates targeting an enzyme conserved either over all bacterial kingdom, over a defined bacterial group such as coliform bacteria or over a defined species of bacteria such as Escherichia coli with either a chromophore or a fluorophore. If a targeted group of microorganisms is present, the enzyme is present inducing the chemical reaction of substrate utilization and liberation of either the fluorophore or the chromophore. The fluorophore or the chromophore are thus accumulating during the enzyme reaction. The chromophore induces the color change of the medium upon liberation and accumulation and the fluorophore induces the increase of intensity of fluorescence upon liberation and accumulation. A number of selective differential chromogenic media have been developed for the detection of E. coli and coliform bacteria and for the detection of enterococci such as AquaChrom™ECC and AquaChrom™Enterococcus from ChromAgar, COLILERT® and ENTEROLERT® from Idexx or EC Blue™ from HyServe. These selective chrmomogenic media contain selective chromogens. Selective chromogens are substrate analogues—the substrate connected to a chromophore—which are cleaved by an enzyme present only in the targeted bacterial group. If the bacterial group and thus the enzyme are present in the sample, the chromophore will be cleaved of and it will accumulate. The resulting unbound chromophore will enable the specific colouring of the sample as a consequence of its specific absorption/emission spectra.

A number of different methods for utilising such media for the MPN have been developed. One of them is the Idexx Ouantitray™ system (U.S. Pat. No. 5,753,456). The system is based on two different trays either a tray of 51 wells of equal volume size or the 97 well tray with two different volume sizes. Also, a number of different patent literature deal with modifications of MPN-based methods and corresponding sample holding devices for bacterial detection, such as US 2017/0240949 A1; US 2017/0247737 A1; WO 2016/051167 A1; US 2013/0189770 A1; US 2010/0136608 A1; US 2010/0273209 A1; US 2005/0048597 A1; US 6,509,168 B2; U.S. Pat. Nos. 6,696,286 B1; 6,365,368 B1; 6,492,133 B1; 6,190,878 B1, 4,868,110; UK Pat Ap. GB 2 106 539.

Compartmentizations of prior art sample holders in the patent literature include for instance: a microwell plate containing from 0.1-100 μL of sample with the reagent (U.S. Pat. No. 6,190,878 B1); an MPN strip composed of one 50 mL, five 10 mL, and five 1 mL compartments (UK Pat Ap. GB 2 106 539); a sample holder composed of five wells of 10 mL volume, five of 1 mL volume and five of 0.1 mL volume along with a system of sample distribution between the wells (US 2013/0189770 A1); a sample holder which distributes the sample into discrete compartments via a capilary flow though radially organized capilary channels (US 2005/0048597 A1); a sample holder (also known as Simplate™) is composed of a round incubation plate divided into a plurality of at least 20 recessed wells (U.S. Pat. No. 6,509,168 B2); sample holders composed of sets of microcompartments with volume sizes ranging from 0.010 μL-25 μL (U.S. Pat. No. 6,696,286 B1).

All of the above-mentioned inventions have certain limitations. One limitation often is the actual complexity of use. Filter-based approaches require additional rinsing and analytical steps after sample filtration to obtain the desired most probable concentration of bacteria, respectively the most probable number of bacteria per mL of sample. CO₂-based detection methods require plural apparati installed for the actual quantification of microorganisms in the sample; and CO₂ alone does not indicate whether the microorganism is a prokaryote or an eukaryote, while in some occasions, however, identification of specific bacterial groups or species are required.

As far as sample holders are concerned, the present inventors have identified the statistical nature underlying the MPN method as a main problem, in particular in view of safeguarding a certain desirable confidence interval (CI), which preferably should be at 95% CI. Specifically, according to a likelihood function of the statistical analysis underlying the MPN method—describing the probability of all outcomes according to a defined concentration value (μ value)—not all outcomes are equally probable for a certain μ value. In the case of the MPN method, basically a higher number of μvalue estimates can be obtained with a larger number of possible outcomes (a broader μestimate range), and likewise a larger number of possible outcomes can basically be obtained with a larger number of total compartments of the sample holder (because a larger theoretical number of outcomes is possible). Ideally, most accurate results would theoretically be obtained if infinite number of dilutions were made or if the sample would be distributed in an infinite number of compartments with infinitely small volumes (then the MPN estimate would equal the actual number of bacterial cells in the sample), which however is practically impossible since it would require volumes of compartments practically in the range of bacteria size and the results would be impossible to interpret. It is therefore the challenge to find a compromise between conflicting goals and thus to provide a balance of simplicity on the one hand and statistical accuracy and predictability on the other hand

### SUMMARY OF THE INVENTION

It is therefore an object of the present invention to provide a method for detection and/or quantification of microorganism in a liquid sample, which method allows easy performance and simple detection systems as well as associated simplified sample holders and processing, however without detriment to obtain reliable results of microorganism detection and enumeration.

The object is solved by the method for detection and/or quantification of microorganism in a liquid sample as defined in claim 1, as well as by the provision of a sample holder as defined in claim 11. Preferred embodiments are set forth in respective dependent claims to claims 1 and 11, respectively. The present invention also provides a kit of parts as defined in claim 21, and also a system as defined in claim 21.

In the method according to the present invention, different volumes—which are present in the compartments of the sample holder—follow a linear distribution pattern, also meant to represent a linear regression pattern. Alternatively to differently distributed volumes following linear distribution pattern, but following the same principle of linear regression, the method may undertake the step of diluting the liquid sample into a number of samples of different dilutions by a dilution factor following a linear distribution (or linear regression) pattern, and in principle introducing the different dilutions (following the linear distribution pattern) into a sample holder (in an alternative embodiment) with multiple possible repetitions of the same dilution. In line with this common principle, in reality an optimal model was found which incorporates a balance between statistical relevance and ease of result interpretation—a simplified method that provides statistically representative results in the predicted concentration range of bacteria, which at most can be expected in a typical liquid sample at issue. The method particularly provides statistically representative results in a relevant concentrational range of microorganisms between 0 CFU (Colony Forming Unts) per 100 mL sample and less than 100 CFU/100 mL of sample, preferably at most 60 CFU per 100 mL of sample. For instance, this concept takes into account an expected concentrational range between 10 and 100 CFU per 100 mL when analysing drinking water, and an expected concentrational range being 10-times greater, from 100-1000 CFU per 100 mL, when analysing wastewater—which original situation however can be easily addressed by appropriate preparation steps, e.g. subjecting drinking water to the analysis method undiluted, while subjecting waster water or industrially born water in a prior appropriate dilution before being subjected to the analysis method.

Based on the principle of presenting a linear distribution pattern, the sample holder of the present invention can be embodied on various embodiments. With multiple embodiments of the sample holder, there are also multiple embodiments of the utilization of the sample holder for the MPN (»Most Probable Number«) Method for the detection and enumeration of bacteria in a liquid sample of volume not less than 100 mL, preferably about 100 mL. The liquid sample may be selected from, for example, drinking water, wastewater, industrial process water, bathing water, recycled wastewater and surface or natural water. All embodiments for the utilization of the sample holder are based on an original liquid sample, to which a selective lyophilized medium may be added and solubilized in the sample. The original sample (containing a certain concentration and a certain number of bacteria) with the solubilized medium is then distributed between the compartments. There is a known total number of compartments following the linear regression scheme. The compartments have known (defined) volumes, a known (defined) number of different volumes following the pattern of linear regression, which in a preferred embodiment is at least five-fold with five different volumes in the increasing linear regression set respectively x, 2x, 3x, 4x and 5x, more preferably eight-fold with eight different volumes in the increasing linear regression set respectively x, 2x, 3x, 4x, 5x, 6x, 7x, and 8x and upto twenty-fold, with twenty different volumes in the increasing linear regression set respectively x, 2x, 3x, 4x, 5x, 6x, 7x, 8x, 9x, 10x, 11x, 12x, 13x, 14x, 15x, 16x, 17x, 18x, 19x and 20x (wherein the volume x represents the smallest volume in the linear regression set), and a known (defined) number of repetitions of the same volume, wherein this number of repetitions is at least three, preferably three. Based on the defined number of different volumes and the defined number of the repetitions of the same volume, the total number of compartments may be deduced in a simplified, yet sufficiently accurate system; accordingly the total number of compartments is preferably no less than 15, more preferably at least 24 (corresponding to 8x times 3) and at most 60 (corresponding to 20x times 3). In the particularly preferred embodiment, the sample holder is composed of 24 compartments with eight different volumes following the pattern of linear regression of V₁=x, V₂=2x, V₃=3x, V₄=4x, V₅=5x, V₆=6x, V₇=7x and V₈=8x or if x is termed as the smallest volume V₁, V₁=V₁, V₂=2V₁, V₃=3V₁, V₄=4V₁, V₅=5V₁, V₆=6V₁, V₇=7V₁ and V8=8V₁. As the sum of all of the internal volumes of the compartments is no less than 100 mL or preferably about 100 mL the different volumes following the linear distribution pattern are V₁=0.926 ml, V₂=1.852 ml, V₃=2.778 ml, V₄=3.704 ml, V₅=4.63 ml, V₆=5.556 ml, V₇=6.481 ml and V₉=7.407 ml. Also, in a preferred embodiment, the sample holder is of hydrophobic material—hydrophobic plastic material or other hydrophobic material, capable of retaining fractions of the liquid sample in accordance with the laws of surface tension and enables that no incidence and flow of water between neighbouring compartments occurs, or of plastic material with a hydrophobic coating which coats at least at a part of or all of the surface facing the holder inner space. Also, in a preferred embodiment, the sample holder is of cylindrical shape and also the compartments of the sample holder are of cylindricall shape. In other embodiments, the shape of the sample holder may be rectangular, spherical, or other shape, and/or may be designed with various dimensions in terms of height and/or width (or radius). In other embodiments, also the shape of the compartments may be rectangular, spherical, or other shape, and/or may be designed with various dimensions in terms of height and/or width (or radius), however in such a way that the volume of each compartment remains unchanged over all different volumes (following the linear pattern) and over each of the repetitions of the same volume. In a preferred embodiment, the compartments are organized in a conical spiral order with a defined distance between the compartments and a defined curvature (height distance between the compartments). In a prefered embodiment, the compartments are organized in a counter clockwise helix moving downward towards the center of the sample holder. In the preferred embodiment shown in FIGS. 1, 2A, 2B and 3, the volumes are organized in triplets with the outermost triplet starting the counter clockwise helix the triplet of volume V₈. At the volume triplet, V₄, the helix turns to a clockwise helix moving again downward towards the center of the sample holder. In other embodimients, the curvature of the spiral may differ and the neighbouring compartments may be on different height differences. Also, in other embodiments, the distance between neighbouring compartments may differ. Also, in other embodiments, the flux of the helix may be either counter clockwise or clockwise moving either downwards towards the center or downwards from the center towards the edge of the sample holder. In a prefered embodiment, the compartments are connected via a central channel enabling a harmonized flux of the liquid sample. In other embodiments, the central channel may be of different dimensions (depth and/or width). In a further preferred embodiment, an additional compartment is added to the model to compensate for the error in measuring and sampling of the 100 mL of the water sample. In a particular embodiment, the total inner volume of all of the compartments (the summ of the internal volumes of all of the compartments) is no less than 100 mL, preferably is 100 mL. In addition, a conical shaped plastic module may be connected to the first compartment of the model to enable a more harmonized water flux and further distributions into following compartments. The statistical calculation of the estimate of the most probable number (MPN) of bacteria per unit volume is derived from the outcome of how many compartments of the same volume are positive from all of the repetitions of the same volume and over all different volumes (how many compartments of the same volume out of all repetitions of the same volume contain at least one microorganism) and the calculation is based on equating the first derivative of the natural logarithm of the likelihood function to zero. Simultaneously, the estimate of the variance of the MPN estimate and the estimate of the standard deviation of the MPN estimate are calculated from the second derivative of the likelihood function and from the estimation of the standard deviation of the MPN estimate, the estimate of the standard deviation of the natural logarithm of the MPN estimate is calculated by dividing the estimate of the standard deviation of the MPN estimate with the MPN estimate. Based on a desirably applied 95% confidence, confidence intervals are then calculated from the estimate of the most probable number and the estimate of the standard deviation of the natural logarithm of the MPN (Most Probable Number) estimate, showing the intervals of concentrations of microorganisms on which there is 95% percent chance that the actual number of microorganisms per unit volume will be. By way of example: for the preferred embodiment composed of 24 wells with eight different volumes following a linear correlation pattern with each different volume present in triplicates, at lower estimates of MPN for example 1 cell per 100 mL of water sample, the estimate of the standard deviation of the natural logarithm of the MPN estimate limits to or practically reaches the value 1. With increasing estimates of MPN, the estimate of the standard deviation of the natural logarithm of the MPN estimate decreases to value 0.4 at the estimate of the MPN 10 CFU/100 mL and to value of 0.3 at the estimate of the MPN 30 CFU/100 mL, then stabilizes and is still at around 0.3 at the estimate of MPN 40 CFU/100 mL of sample. With further increasing the estimates of MPN, the standard deviation ranges in the values between 0.3 and 0.35. Calculating the lower and upper limits of 95% confidence intervals; at the estimate of MPN 1 CFU/100 ml of sample, the lower limit of the 95% confidence intervals is 0.001 CFU/mL and the upper limit is 0.07 CFU/mL. This in practice means that the actual number of cells in the respective 100 mL water sample should by calculation vary between 1 and 8 cells per 100 mL of water sample, with the Most Probable Number of cells being 1 per 100 mL of water sample. At the MPN estimate of 10 CFU/100 mL the lower limit of the 95% confidence interval is at approximately 0.04 CFU/mL and the upper limit of the 95% confidence interval is at approximately 0.2 CFU/mL. This in practice means that the actual number of cells in the respective 100 mL water sample should by calculation vary between 4 and 20 cells per 100 mL of water sample, with the Most Probable Number of cells being 10 per 100 mL of water sample. At the MPN estimate 30 CFU/100 mL, the lower limit of the 95% confidence interval is 0.16 CFU/mL and the upper limit of the 95% confidence interval is 0.55 CFU/mL. This in practice means that the actual number of cells in the respective 100 mL water sample should by calculation vary between 16 and 55 cells per 100 mL of water sample with the Most Probable Number of cells being 30 per 100 mL of water sample. At the MPN estimate 40 CFU/100 mL the lower limit of the 95% confidence interval is at 0.2 and the upper limit of the 95% confidence interval is at 0.7. This in practice means that the actual number of cells in the respective 100 mL water sample should by calculation vary between 20 and 70 cells per 100 mL of water sample with the Most Probable Number of cells being 40 per 100 mL of water sample. However, one should always also observe the total number of positives out of 24 compartments in determining the minimum possible number of cells (lower limit) present in the sample as every positive compartment indicates at least a single present cell in the respective positive compartment. Also, in other embodiments an automatized form of the sample holder may be developed enabling an automatized detection of positive compartments of a defined volume out of all repetitions of the defined volume and over all different volumes, an automatized calculation of the estimate of the Most Probable Number from the outcome and a further automatized generation or calculation of the upper and lower limits of the 95% confidence intervals. The utilization of the sample holder for the MPN method or the detection and enumeration of bacteria in the liquid sample relies on the feature of the statistical calculation of the most probable concentration of the microbiological sample (most probable number of microorganisms per mL of sample). The most probable number of microorganisms can be derived from every possible combination of dilution and volume of the respective sample aliquote after partitioning from the maximum of the natural logarithm likelihood function defined by two probability functions, namely the Poisson distribution function and the binomial distribution function; the maximum likelihood occurs, when the first derivative of the natural logarithm of the likelihood function equals zero. The ratio between the volumes does not need to be constant, thus it does not need to be 2 nor 10. Different number of compartments with a specific combination of dilution and volume may be applied. The present invention benefitially relies on the improvement of the statistics in terms of confidence intervals at smaller numbers of microorganisms (respectively less than 100 CFU/100 mL or preferrably between 0 CFU/100 mL and 60 CFU/100 mL) of water sample (the 95% confidence intervals are narrow at smaller MPN estimates, for example from 0 CFU/100 mL to 10 CFU/100 mL and further linearly increase with increasing values of MPN estimates, thus a statistically relevant estimate of the MPN value can be concluded at actual numbers of microorganisms between 0 CFU/100 mL to 60 CFU/100 mL), which means that the statistics are improved for the exact application of the model. Narrow intervals are prefered at smaller numbers of microorganisms (<100 CFU/100 mL, preferably between 0 CFU/100 mL to 60 CFU/100 mL) as the prefered embodiments for the model are for drinking water, where there are usually very low concentrations of microorganisms (between 0 CFU/100 mL to 60 CFU/100 mL), or where simply a negative result (absence of microorganisms) needs to be confirmed. This is because the different volumes of the compartments of the respective sample holder follow a linear regression pattern. The basic principle of underlying the MPN method, and correspondingly structure the sample holder with a corresponding number of compartments, to follow a linear regression pattern allows to apply always an optimum between ease and simplicity of method processing and sample holder structuring, and reliability of the obtained results of microorganism detection and enumeration.

### BRIEF DESCRIPTION OF THE DRAWINGS

In the drawings:

FIG. 1 shows a sample holder according to one embodiment of the present invention in a perspective view from topside;

FIGS. 2A and 2B show the sample holder according to the embodiment of FIG. 1 in a plan view (FIG. 2A) and in a sectional view (FIG. 2B); and

FIG. 3 shows the sample holder according to the embodiment of FIG. 1 in a perspective side view, partly sectioned.

FIG. 4 shows results of confidence intervals upon using a linear distribution of the volumes according to the MPN method according to the present invention. Horizontal axis represents the MPN estimate upon a certain outcome and the vertical axis represents the actual value of the number of bacteria per mL of the sample. The vertical lines represent confidence intervals.

FIG. 5 shows results of confidence intervals upon an exponential distribution of the volumes for comparison. Horizontal axis represents the MPN estimate upon a certain outcome and the vertical axis represents the actual value of the number of bacteria per mL of the sample. The vertical lines represent confidence intervals.

FIG. 6 shows the correlation curve established in Example 2 between the actual number of bacteria and optical density.

FIG. 7 shows the dilutions and preparation of samples used in Example 3, and

FIG. 8 shows the dilutions and preparation of samples used in Example 4.

### DESCRIPTION OF PREFERRED EMBODIMENTS

This invention relates to a multiplicity of the embodiments of the “Most Probable Number” (MPN) based method and of the sample holder, which is utilized for the MPN for identification of microorganisms in drinking and wastewater samples.

The invention relates to a sample holder, constructed of a distinct number of compartments, namely at least 15, preferably 24 and at most 60. The compartments are of different volumes following a linear distribution pattern. Each different volume may be present in a multitude of repetitions

Appropriately, the linear distribution pattern is at least 5-fold, namely with five different volumes following the increasing linear distribution set of x, 2x, 3x, 4x and 5x, preferably and preferably provides the linear distribution pattern of up to 20-fold, namely with 20 different volumes following the linear distribution of x, 2x, 3x, 4x, 5x, 6x, 7x, 8x, 9x, 10x, 11x, 12x, 13x, 14x, 15x, 16x, 17x, 18x, 19x and 20x. [17] Accordingly, the sample holder may be structured to provide a corresponding number of different compartments, the different compartments being sized to respectively define the linear distribution pattern. Thus, the sample holder and thereby the method can beneficially use anyone of the following linear volume distribution patterns with different volumes following the increasing linear distribution of the linear set:
1x-, 2x-, 3x-, 4x- and 5x-fold volume distributions;
1x-, 2x-, 3x-, 4x-, 5x-, 6x-, 7x- and 8x-fold volume distributions;
1x-, 2x-, 3x-, 4x-, 5x-, 6x-, 7x-, 8x- and 9x-fold volume distributions;
   and subsequent linear volume distributions correspondingly increased by a further number, up to 1x-, 2x-, 3x-, 4x-, 5x-, 6x-, 7x-, 8x-, 9x-, 10x-, 11x-, 12x-, 13x-, 14x-, 15x-, 16x-, 17x-, 18x-, 19x- and 20x-fold volume distributions. Preferably the linear distribution pattern consists of lx-, 2x-, 3x-, 4x-, 5x-, 6x-, 7x- and 8x-fold volume distributions.

During the practical utilization, it is useful and provides more reliable results when the number of volume distributions is adapted to the number of expected microorganisms depending on the type of original sample, for example drinking water, waste water, industrial process water, bathing water and surface or natural water. Accordingly, a relatively low number of linearly distributed different volumes in the linear distribution set in the range of, for example, at least 5-fold and at most 12-fold, expecially in case of 8-fold linearly distributed volume proportions of different volumes, is suitable for relatively pure liquids such as drinking water. On the other hand, if a relatively impure liquid sample is to be analyzed, such as waste water or industrial water, a relatively large number of linearly distributed different volumes in the linear distribution set in the range of, for example, at least 12-fold and at most 20-fold is more preferred.

Considered alternatively or in combination with such adaptation to the type of liquid to be analyzed, it is preferred that the liquid sample subjected to method step (a) contains less than 100 CFU microorganisms per 100 mL, preferably at most 60 CFU microorganisms per 100 mL. Accordingly, if the liquid sample is drinking water, the drinking water beneficially does not have to be diluted before subjecting the drinking water sample to step (a). If on the other hand the liquid sample is obtained from wastewater, industrial processing water and/or natural water, said liquid sample is preferably diluted once before subjecting said water sample to step (a), preferably is diluted at least 1:10, more preferably is diluted 1:100.

In a preferred embodiment common to any of modifications and embodiments of the sample holder, the respective compartment defining each volume of the linear distribution pattern is present in triplicate of a same volume each. Accordingly the number of the repetitions of the same volume in the linear distribution set of different volumes is at least three, preferably is three. This leads to substantially enhanced and statistically more reliable results. Accordingly, depending on the aforementioned number of linear distributions, the respectively x-fold volume dilutions are present during the analysis method, and is correspondingly present in the sample holder, in a three-fold total number. This means that, optionally, in total 15 compartments are formed in case of lx-, 2x-, 3x-, 4x- and 5x-fold linear distributions of different volumes; in total 24 compartments are formed in case of lx-, 2x-, 3x-, 4x-, 5x-, 6x-, 7x- and 8x-fold linear distributions of different volumes; and so on with correspondingly increasing numbers of linerar regression, up to forming 60 compartments in case of lx-, 2x-, 3x-, 4x-, 5x-, 6x-, 7x-, 8x-, 9x-, 10x-, 11x-, 12x-, 13x-, 14x-, 15x-, 16x-, 17x-, 18x-, 19x- and 20x-fold linear distributions of different volumes. [21] In particularly exemplified embodiments and related variations, the sample holder may have an outer shape selected from a preferably cylindrical outer shape to other shapes such as a spherical, a rectangular outer shape or other possible shapes and/or other dimensions in terms of height and/or width (or radius). Independent from such outer shape, the shape of each compartment may be appropriately selected, e.g from preferably cylindrical compartments, rectangular compartments, spherical compartments or other shape and/or other dimensions in terms of height and/or width (or radius), however in such a way that the volume of each compartment remains unchanged over all different volumes, following the linear pattern and over each of the repetitions of the same volume. Preferably, the sample holder comprises cylindrical compartments as the compartments, which respectively define, said linear volume distribution pattern, more preferably in combination with the sample holder having itself a cylindrical outer shape comprising such cylindrical compartments.

In a particular embodiment, the sample holder is arranged to define a specific total volume of not less than, preferably about 100 mL for holding the liquid sample. When such a specific total volume for holding the sample is given as V=100%, and when according the above mentioned preferred embodiment the respective compartment defining each volume of the linear distribution pattern is present in triplicate, then for each of three compartments of the linear distribution pattern the 1x unit volume is 0.926% of V, the 2x-fold unit volume is 1.852% of V, the 3x-fold unit volume is 2.778% of V, the 4x-fold unit volume is 3.704% of V, the 5x-fold unit volume is 4.63% of V, the 6x-fold unit volume is 5.556% of V, the 7x-fold unit volume is 6.481% of V, and the 8x-fold unit volume is 7.407% of V, wherein each %-volume indication encompasses a ±10% volume tolerance range, preferably a ±5% volume tolerance range, more preferably a ±1% volume tolerance range. As mentioned, it is preferred in terms of standardization that the liquid sample subjected to step (a) in the method, or correspondingly the total volume holding capacity for the sample in the holder, has a volume of no less than 100 mL and more preferably is 100 mL and accordingly the aforedefined V=100% is 100 mL. [23] In one embodiment, the sample holder is preferably constructed to be composed of 24 compartments, divided into eight triplets, thus with three repetitions of eight different volumes. The volumes following a linear distribution pattern then comprise; V₁=V₁ (the volume of the smallest triplet), V₂=2V₁, V₃=3V₁, V₄=4V₁, V₅=5V₁, V₆=6V₁, V₇=7V₁ and V₈=8V₁ or x (volume of the smallest triplet), 2x, 3x, 4x, 5x, 6x, 7x and 8x. The volumes of the model are preferably: V1=0.926 mL, V2=1.852 mL, V3=2.778 mL, V4=3.704 mL, V5=4.63 mL, V6=5.556 mL, V7=6.481 mL and V8=7.407 mL. The total inner volume of all compartments is no less then 100 mL, preferably about 100 mL, which complies with ISO standards ISO 7899-1, ISO 9308-3 and ISO 9308-2.

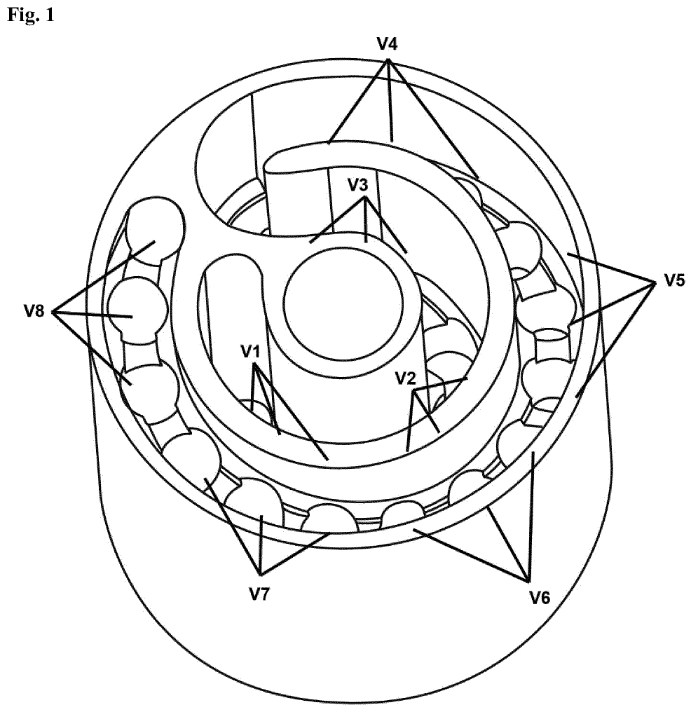

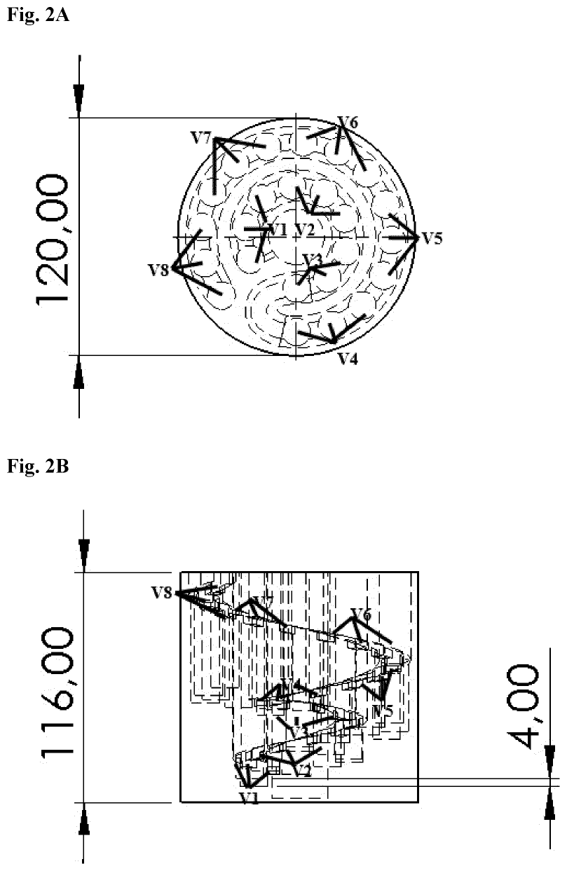

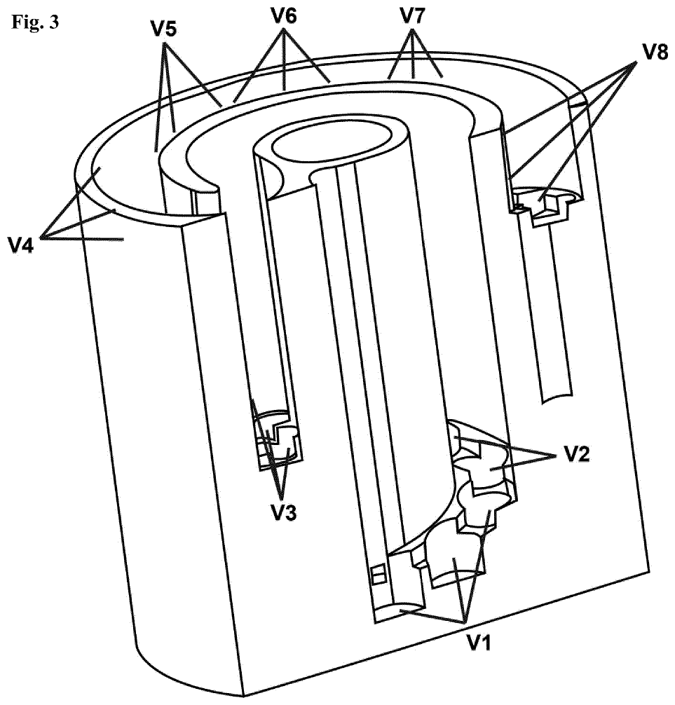

In one embodiment, cylindrical columns form the compartments in the sample holder. Examples of such an embodiment is shown in FIGS. 1-3. The cylindrical compartments comprise, each in triplicate, V₁ making up the smallest volume, V₂=2V₁, V₃=3V₁, V₄=4V₁, V₅=5V₁, V₆=6V₁, V₇=7V₁ and V₈=8V₁ to thereby represent the lx, 2x, 3x, 4x, 5x, 6x, 7x and 8x fold linear volume distribution. In the prefered embodiment, the cylindrical compartments are organized in a conical spiral order with a defined distance between the compartments and a defined curvature (height distance between the compartments, which preferably may be around 5 mm). In the prefered embodiment, the compartments are organized in a counter clockwise helix moving downward towards the center of the sample holder. The volumes are organized in triplets with the outermost triplet (FIGS. 1, 2A, 2B and 3) starting the lefthand helix the triplet of volume V₈. At the volume triplet, V₄, the helix turns to a clockwise helix moving again downward towards the center of the sample holder. In other embodiments, the curvature of the spiral may differ and the neighbouring compartments may be on different height differences. Also, in other embodiments, the distance between neighbouring compartments may differ. Also, in other embodiments, the flux of the helix may be either counter clockwise or clockwise moving either downwards towards the center or downwards from the center towards the edge of the sample holder. The total height of the model may be, for example, around 116 mm and the total diameter of the model may be, for example, 120 mm (FIGS. 2A and 2B). In the preferred embodiment, the compartments are connected via a central channel, preferably of 5 mm width enabling a harmonized flux of the liquid sample. In other embodiments, the central channel may be of different dimensions (depth and/or width). In the preferred embodiment, an additional compartment is added to the model to compensate for the error in measuring and sampling of the 100 mL of the water sample.

The outline of the preferred embodiment of the model is related to a harmonized distribution of sample in the compartments in which the content of the compartments do not contact each other. Thus, during filling the sample into the sample holder, it is starting with the first compartment with volume V₈ (FIG. 2); when the compartment is filled; the water continuously flows in the next compartment. When the second compartment is filled, the water proceeds in the third compartment, and so on. In the end there is no possibility of the content of the compartment being in contact between different compartments.

While the model defines relative volume distributions, the absolute volume per compartment in the sample holder embodiment with cylindrical compartment shape depends on the radius or diameter of each cylindrical compartment; correspondingly the heights of each cylindrical compartment also follow a linear pattern.

In a non-limiting example, the diameter is equal or about 14 mm, thus the heights also following a linear pattern respectively are: h₁=6.01 mm, h₂=12.03 mm, h₃=18.05 mm, h₄=24.06 mm, h₅=30.08 mm, h₆=36.09 mm, h₇=42.10 mm and h₈=48.12 mm. The difference in height between neighbouring compartments is preferably around 5mm. The total height of the model is around 116 mm and the total diameter of the model is 120 mm.

In a particular embodiment, the volume of the smallest of the eight volume triplicates (V₁) can be calculated as: 3V₁+3x2V₁+3x3V₁+3x4V₁+3x5V₁+3x6V₁+3x7V₁+3x8V₁=100 mL.

Then, as a total volume capacity for holding the sample, there are present 108x V1=100 mL, V1=0.926 mL=0.926 cm³

In yet another aspect the volumes of the eight triplicates is indicated in Table 1 below:

TABLE 1

| | Volume | Number of repetitions |
|---|---|---|
| Designation of volume | (mL oz cm³) | of the same volume |
| V1 | 0.926 | 3 |
| V2 | 1.852 | 3 |
| V3 | 2.778 | 3 |
| V4 | 3.704 | 3 |
| V5 | 4.63 | 3 |
| V6 | 5.556 | 3 |
| V7 | 6.481 | 3 |
| V8 | 7.407 | 3 |

Also, in the prefered embodiment of the sample holder outline, the compartments are of cylindrical shape with equal diameter, preferably around 14 mm but with heights corresponding the preferred volume of each triplicate. The heights can be calculated as:

h=V/πr², where h is the heiht of each compartment triplicate and r is the radius of each compartment.

The heights are then preferably as indicated below in Table 2, weherein the height values are approximated values:

TABLE 2

| | | Number of repetitions |
|---|---|---|
| Designation of height | height (mm) | of the same volume |
| h₁ | 6.01 | 3 |
| h₂ | 12.03 | 3 |
| h₃ | 18.05 | 3 |
| h₄ | 24.06 | 3 |
| h₅ | 30.08 | 3 |
| h₆ | 36.09 | 3 |
| h₇ | 42.10 | 3 |
| h₈ | 48.12 | 3 |

Also, in the preferred embodiment, the sample holder is of hydrophobic material—hydrophobic plastic material or other hydrophobic material, capable of retaining fractions of the liquid sample in accordance with the laws of surface tension and enables that no incidence and flow of water between neighbouring compartments occurs or of plastic material with a hydrophobic coating, coating at least at a part of or all of the surface facing the holder inner space. By this hydrophobic plastic material or other hydrophobic material it is possible to effectively retain fractions of the liquid sample in accordance with the laws of surface tension and to allow no incidence and no flow of water between neighbouring compartments to occur.

In another aspect of the prefered embodiment, a conical shaped plastic module may be connected to the first compartment of the model to enable a more harmonized water flux and further distributions into following compartments.

In other embodiments of the substrate holder, the number of compartments may vary maintaining the linear pattern and the 100 mL total internal volume. The numbers of compartments may vary from at least 9 to about 60 or more. With 9 compartments in total, customers willl gain a rough estimate of the most probable at extremely low numbers of bacteria. Another precaution is with 60 compartments is that the volume of the smallest compartment triplicate would be 158 μL (158.7 μL) which indicates a higher probability of technical error in volume upon manufacture. Accordingly, a total of 24 compartments is particularly prefered.

In modified embodiments, other dimensions of the substrate holder regarding total diameter, total height, and radius and height of each compartment, width of the channel and difference in the height between neighbouring compartments can be varied.

In other embodiments, the outer shape of the model is not limited to a cylindrical shape, and the shape of the compartments is not limited to be cylindrical, although it is prefered because of the harmonized flux of the water upon filling the compartments.

In another embodiment of the present invention, the sample holder has a round, petri-dish like structure. The petri dish like structure is composed of two parts. One part is the lid of the sample holder. The other part is a disc-like structure, separated into compartments in a specific manner. The disc is divided into multiple circular sections of angles 10°, 20°, 30°, 40°, 50°, 60°, 70° and 80°. Each circular section is further divided into three compartments by two circular rings. In an alternative embodiment of the sample holder having a round, petri-dish like structure, the compartments-forming disc is divided into multiple circular sections of angles 8°, 16°, 24°, 32°, 40°, 48°, 56°, 64° and 72°. Also in this outer shape configuration of the sample holder, each compartment volume is present in triplicate of a same volume each.

In another embodiment of the present invention, the sample holder has a rectangular outer shape whose compartments divide the sample holder into rectangular sections having said number of compartments respectively defining the linear volume distribution. Again, preferably each compartment volume is present in triplicate of a same volume each.

In prefered embodiments of the sample holder in a disc like structure and/or in the rectangular outer shape structure, the sum of the volume of the compartments is no less than 100 mL and more preferably is 100 mL, which is in accordance to the ISO standards.

In one aspect, the volumes of the compartments, comprising the same circular section are equal.

In another aspect the respective volumes of each compartments comprising each of the eight circular sections follow the pattern x, 2x, 3x, 4x, 5x, 6x, 7x and 8x.

In the preferred embodiment, where the sum of the volumes of the compartments is no less than 100 mL, the volumes of the compartments are as follows: 0.925 mL for each of the three compartments of the 10° circular section, 1.852 mL for each of the three compartments of the 20° circular section, 2.778 mL for each of the three compartments of the 30° circular section, 3.704 mL for each of the three compartments of the 40° circular section, 4.63 mL for each of the three compartments of the 50° circular section, 5.556 mL for each of the three compartments of the 60° circular section, 70° so 6.481 mL for each of the three compartments of the 70° circular section and 7.407 mL for each of the three compartments of the 80° circular section.

The height of the petri dish-like sample holder is not relevant, nor is the thickness of the edges forming each compartment.

In the prefered embodiment, the sample holder is of a plastic material suitable for holding aliquotes of the liquid sample according to the physics of surface tension.

In the preferred embodiment, the sample holder is related to the MPN method of detection of microorganisms in drinking water samples as in the ideal case, the mean total number of bacteria present in the water sample is between 30-40 CFU/100 mL of water sample. The regulation requires less than 100 CFU/mL of water sample in heterotrophic plate counts after incubation at 36° C. and no unusual changes in heterotrophic plate counts at 22° C. After calculating the 95% confidence intervals for each statistical assessment of the most probable concentration of bacteria, the method is very accurate for total concentrations of bacteria up to 60 CFU/100 mL of drinking water sample. For the preferred embodiment composed of 24 wells with eight different volumes following a linear correlation pattern with each different volume present in triplicates, at lower estimates of MPN for example 1 cell per 100 mL of water sample, the estimate of the standard deviation of the natural logarithm of the MPN estimate limits to or practically reaches the value 1. With increasing estimates of MPN, the estimate of the standard deviation of the natural logarithm of the MPN estimate decreases to value 0.4 at the estimate of the MPN 10 CFU/100 mL and to value of 0.3 at the estimate of the MPN 30 CFU/100 mL, then stabilizes and is still at around 0.3 at the estimate of MPN 40 CFU/100 mL of sample. With further increasing the estimates of MPN, the standard deviation ranges in the values between 0.3 and 0.35. Calculating the lower and upper limits of 95% confidence intervals; at the estimate of MPN 1 CFU/100 ml of sample, the lower limit of the 95% confidence intervals is 0.001 CFU/mL and the upper limit is 0.07 CFU/mL. This in practice means that the actual number of cells in the respective 100 mL water sample should by calculation vary between 1 and 8 cells per 100 mL of water sample, with the Most Probable Number of cells being 1 per 100 mL of water sample. At the MPN estimate of 10 CFU/100 mL the lower limit of the 95% confidence interval is at approximately 0.04 CFU/mL and the upper limit of the 95% confidence interval is at approximately 0.2 CFU/mL. This in practice means that the actual number of cells in the respective 100 mL water sample should by calculation vary between 4 and 20 cells per 100 mL of water sample, with the Most Probable Number of cells being 10 per 100 mL of water sample. At the MPN estimate 30 CFU/100 mL, the lower limit of the 95% confidence interval is 0.16 CFU/mL and the upper limit of the 95%confidence interval is 0.55 CFU/mL. This in practice means that the actual number of cells in the respective 100 mL water sample should by calculation vary between 16 and 55 cells per 100 mL of water sample with the Most Probable Number of cells being 30 per 100 mL of water sample. At the MPN estimate 40 CFU/100 mL the lower limit of the 95% confidence interval is at 0.2 and the upper limit of the 95% confidence interval is at 0.7. This in practice means that the actual number of cells in the respective 100 mL water sample should by calculation vary between 20 and 70 cells per 100 mL of water sample with the Most Probable Number of cells being 40 per 100 mL of water sample. However, one should always also observe the total number of positives out of 24 compartments in determining the minimum possible number of cells (lower limit) present in the sample as every positive compartment indicates at least a single present cell in the respective positive compartment.

Accordingly, it is possible to calculate the 95% confidence intervals at increasing estimates of the MPN. The calculations of the 95% confidence intervals are made from the estimate of the standard deviation of the natural logarithm of the MPN estimates. First, estimates of the standard deviation of the natural logarithm of the MPN estimates are made at increasing MPN estimates. From the calculations of the standard deviations of the natural logarithm of the MPN estimates, 95% confidence intervals are calculated by multiplying the value of the standard deviation of the natural logarithm of the MPN estimate at a respective MPN estimate with the respective MPN estimate. The standard deviation of the matural logarithm of the MPN estimates is a function and the MPN estimate is a function. As a result of the multiplication of two functions the 95% intervals are increasing linearly with increasing values of MPN estimates.

Also in other embodiments an automatized form of the sample holder may be developed enabling an automatized detection of positive compartments of a defined volume out of all repetitions of the defined volume and over all different volumes, an automatized calculation of the estimate of the Most Probable Number from the outcome and a further automatized generation or calcualtion of the upper and lower limits of the 95% confidence intervals.

In the preferred embodiment, the sample holder is related to the MPN method of detection of microorganisms in drinking water samples as in the ideal case, the mean total number of bacteria present in the water sample is between 30-40 CFU/100 mL of water sample. The regulation requires less than 100 CFU/mL of total bacterial in the water sample in heterotrophic plate counts after incubation at 36° C. and no unusual changes in heterotrophic plate counts at 22° C. After calculating the 95% confidence intervals for each statistical assessment of the most probable concentration of bacteria, the method is very accurate for total concentrations of bacteria up to 60 CFU/100 mL of drinking water sample.

In another aspect, multiple materials may be used to construct the mentioned sample holder.

In the preferred embodiment, the present invention relates to a MPN method of detection of bacteria, which involves adding a sterile liophylised powdered media to the liqud sample such as the drinking water. The medium is a selective chromogenic medium for the detection of E. coli and other coliform bacteria, containing two different chromogen reagents, one sensitive to E. coli and the other sensitive to other coliform bacteria. The chromogen selective for E. coli targets the E. coli specific β-glucuronidase and has a chromophore linked to a β-D-glucuronide. The chromogen selective for other coliform bacteria alternatively targets β-galactosidase and has a different chromophore attached to a β-D-galactopyranosid. If E. coli and also coliforms other than E. coli are present in a selected compartment, β-glucuronidase is present in the compartment and also β-galactosidase. The enzyme β-glucuronidase will liberate the chromophore attached to β-D-glucuronide and the β-galactosidase will liberate the different chromophore attached to β-D-galactopyranosid. The liberated chromophore attached to β-D-glucuronide will enable medium change to green upon accumulation and the chromophore attached to β-D-galactopyranosid will enable the medium change to yellow upon accumulation. However, the green pigment in the compartment where both E. coli and coliforms other than E. coli are present will overcome the yellow pigment. If only E. coli is present in a compartment, β-glucuronidase is present in the compartment. The enzyme β-glucuronidase will liberate the chromophore attached to β-D-glucuronide and the colour of the compartment will change to green upon accumulation of the chromophore. If coliforms, other than E. coli are present in the compartment, the present enzyme β-galactosidase will liberate the different chromophore attached to β-D-galactopyranosid. This will enable the colour change of the medium to yellow. If other bacteria are present which are neither E. coli nor belong to the group of coliform bacteria, the medium changes to opaque. The output of the method is then defined by the combination of the number of yellow and green compartments of a specific volume out of all repetitions of the same volume and over all different volumes.

Other embodiments of the present invention relate to the detection of E. coli and other coliform bacteria, with the MPN method in the drinking water sample optionally also involve other choices of selective chromogenic media. The method of detection of positives involves a color change of the sample based as a consequence of free chromophore accumulation and the change in absorption/emission spectra of the sample.

Other embodiments of the present invention relate to the detection of E. coli and other coliform bacteria in the drinking water sample with the MPN method may involve adding sterile liophylised fluorogenic (and chromogenic) media to the water sample. This medium containes a monosaccharide connected to a fluorophore and a monosaccharide connected to a chromophore—both target different bacterial groups, either E. coli or other coliform bacteria. In this case, the method of detection of positives involves emmiting a light of a specific wavelength upon excitation with UV light, due to accumulation of a fluorophore.

Also, other embodiments of the present invention may relate to the detection of enterococci in the drinking water sample via the MPN method.

Further embodiments of the present invention relate to the MPN method of detection of enterococci in the drinking water sample may involve adding sterile, liophylised selective chromogenic or fluorogenic media for the detection of enterococci, with chromogens (or substrate analogues) sensitive to enterococci.

Further embodiments of the present invention relate to the detection of other bacterial species, e.g. Legionella sp., Bacillus sp., Pseudomonas sp., and others in drinking water, via the MPN method, which involves a choice of a suitable selective media, either chromogenic or fluorogenic.

Further embodiments of the invention may involve detection of total heterotrophic bacteria or total bacteria in the drinking water via the MPN method, which again involves a choice of suitable medium containing reagents detecting microbial presence.

Other embodiments of the present invention may also relate to the MPN method of detection of microorganisms in wastewater samples, industrial process water samples, bathing water, surface or natural water samples, recycled wastewater samples. Herein, suitable previous dilutions of the sample are suggested in order to reach the optimal range of 95% confidence intervals.

Further embodiments of the present invention may involve detection of E. coli and/or other coliform bacteria, enterococci, total bacteria or other specific bacterial groups in the wastewater sample using either selective fluorogenic, chromogenic or other suitable media in a similar way as with the drinking water samples.

In a further aspect, the present invention provides an automized form of the inventive method in any of the above embodiments, wherein the presence of bacteria is detected by a positive signal in a defined volume, out of all repetitions of the defined volume and over all different volumes. The automatized detection preferably includes an automatized calculation of the estimate of the Most Probable Number from the positive signal results. The method may optionally additionally include a further automatized generation or calculation of the upper and lower limits of the 95% confidence intervals. In another aspect, the present invention provides an automized system comprising the sample holder described above, and a detector for detecting the presence of bacteria accordingly. Suitable means for detection of the positive signals are known. For example, the method may use and the system may comprise an appropriate number of LED light emitters and a corresponding number of sensors sensing the emission of light or fluoresence. For descrition of further features of the inventive method and sample holder reference is made to the above description.

All described embodiments of the present invention may be linked to an automatized method of detection of either color change or fluorescence intensity, further calculation of the estimate of the Most Probable Number out of the outcome of the experiment and calculation of the 95% confidence intervals from the estimate of the standard deviation of the natural logarithm of the MPN estimate and from the MPN estimate.

In all described embodiments, the advantage of the present invention is that very accurate statistical results are provided in the concentration range of microorganisms in drinking water (upto 60 CFU per 100 mL) despite a relatively low number of compartments, because the sizes of volumes follow a linear regression.

### EXAMPLES

### Example 1—Proving the Concept of Better Model Statistics and Providing more Accurate Results Upon Linear Distribution the Volume of Compartments

In this example, two outlines of the model were taken into account. The first one is the outline with linear dustribution of the volumes, as shown in the folowing Table 3.

TABLE 3

| | Volume | Number of repetitions |
|---|---|---|
| Designation of volume | (mL oz cm³) | of the same volume |
| V₁ | 0.926 | 3 |
| V₂ | 1.852 | 3 |
| V₃ | 2.778 | 3 |
| V₄ | 3.704 | 3 |
| V₅ | 4.63 | 3 |
| V₆ | 5.556 | 3 |
| V₇ | 6.481 | 3 |
| V₈ | 7.407 | 3 |

Where V₁ = V₁, V₂ = 2V₁, V₃ = 3V₁, V₄ = 4V₁, V₅ = 5V₁, V₆ = 6V₁,V₇ = 7V₁, V₈ = 8V₁

In a comparison example an exponential distribution of the volumes with constant ratio between adjacent different voumes, as shown in Table 4.

TABLE 4

| | Volume | Number of repetitions |
|---|---|---|
| Designation of volume | (mL oz cm³) | of the same volume |
| V₁ | 0.131 | 3 |
| V₂ | 0.262 | 3 |
| V₃ | 0.524 | 3 |
| V₄ | 1.048 | 3 |
| V₅ | 2.096 | 3 |
| V₆ | 4.192 | 3 |
| V₇ | 8.384 | 3 |
| V₈ | 16.768 | 3 |

Where V₁ = V₁, V₂ = 2V₁, V₃ = 4V₁, V₄ = 8V₁, V₅ = 16V₁, V₆ = 32V₁, V₇ = 64V₁, V₈ = 128V₁

Confidence intervals were calculated with RStudio and ploted in a linear plot. In principle, upon plotting linear plots of confidence intervals, the intervals should increase upon increasing estimate of the must probable number

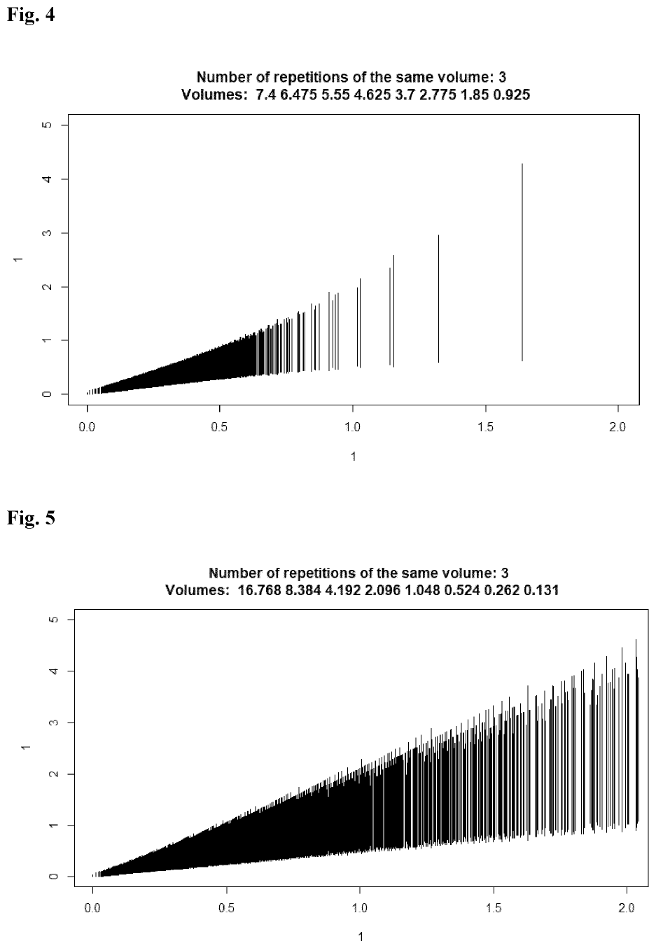

Results can be depicted from FIGS. 4 and 5 for linear and exponential distribution of volumes, respectively.

In case of linear volume distribution, a relatively narrow confidence intervals can be observed up to 0.6 CFU/100 mL of water sampleor 60 CFU/100 mL of water sample, which increase linearly (FIG. 4). If a linear distribution of the volumes intervals is chosen, it is possible to be relatively more flexible regarding the number of compartments, because as can be seen, upon linear distribution, the smallest volume of the model containing 20 different volumes following a linear distribution in triplicates is 130-150 μL, where in case of the exponential volume distribution, the volume 130 μL is reached when the model contains 8 different volumes in triplicates, with different volumes following an exponential distribution. Thus, with the linear distribution volumes decrease more slowly and a sample holder can be arranged with more compartments than upon exponential volume distribution.

In the case of exponential distribution of volumes for comparison (cf. FIG. 5), one can observe broader confidence intervals, which are non-consistent and do not increase linearly, compared to the linear distribution of the model. In the exponential distribution of different volumes, the 95% confidence intervals are howeverdense through a wider range upto 100 CFU/100 ml of water sample. In addition, the confidence intervals do not increase completely linearly, rather, belts of slightly broader intervals can be seen.

From this, it is concluded that linear distribution of volumes, although in a narrower range of bacterial concentration, behaves better.

### Example 2—Creating the Correlation Curve Between the Concentration of E. coli and OD

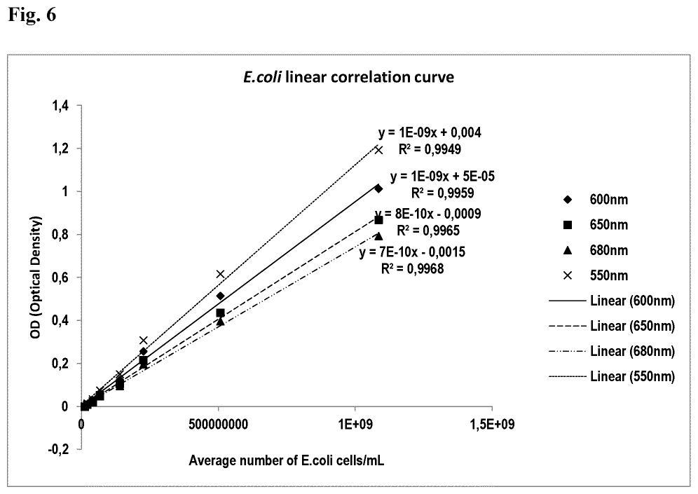

A correlation curve between the actual number of bacteria and optical density was constructed. E. coli was introduced into sterile distilled water via inoculation loop upoto optical density (OD) 1. Thereafter, serial dilutions of 2 were made. Each serial dilution was counted with the Neubauer counting chamber and simultaneously its OD was measured. The below Table 3 and the graph shown in FIG. 6 represent the results with the trendilnes.

TABLE 5

| SAMPLE | 600 nm | 650 nm | 680 nm | 550 nm | N1 | N2 | N3 | N4 | N5 | Average N | CFU/mL |
|---|---|---|---|---|---|---|---|---|---|---|---|
| Sample-9 | 1.0129 | 0.8670 | 0.7924 | 1.1923 | 466 | 496 | 401 | 432 | 376 | 434.2 | 1085500000 |
| Sample-10, | 0.5141 | 0.4358 | 0.3959 | 0.6162 | 251 | 214 | 208 | 203 | 138 | 202.8 | 507000000 |
| Sample-11, | 0.2554 | 0.2161 | 0.1958 | 0.3076 | 92 | 90 | 94 | 89 | 88 | 90.6 | 226500000 |
| Sample-12, | 0.1240 | 0.1052 | 0.0954 | 0.1501 | 65 | 60 | 55 | 49 | 51 | 56 | 140000000 |
| Sample-13, | 0.0607 | 0.0517 | 0.0474 | 0.0743 | 26 | 27 | 27 | 29 | 27 | 27.2 | 68000000 |
| Sample-14, | 0.0264 | 0.0225 | 0.0203 | 0.0333 | 17 | 16 | 14 | 19 | 17 | 16.6 | 41500000 |
| Sample-15, | 0.0099 | 0.0081 | 0.0073 | 0.0139 | 7 | 8 | 8 | 12 | 9 | 8.8 | 22000000 |
| Sample-16, | −0.0004 | −0.0009 | −0.0013 | 0.0014 | 6 | 6 | 4 | 5 | 5 | 5.2 | 13000000 |

### Example 3—Confirmation of the Linear Curve

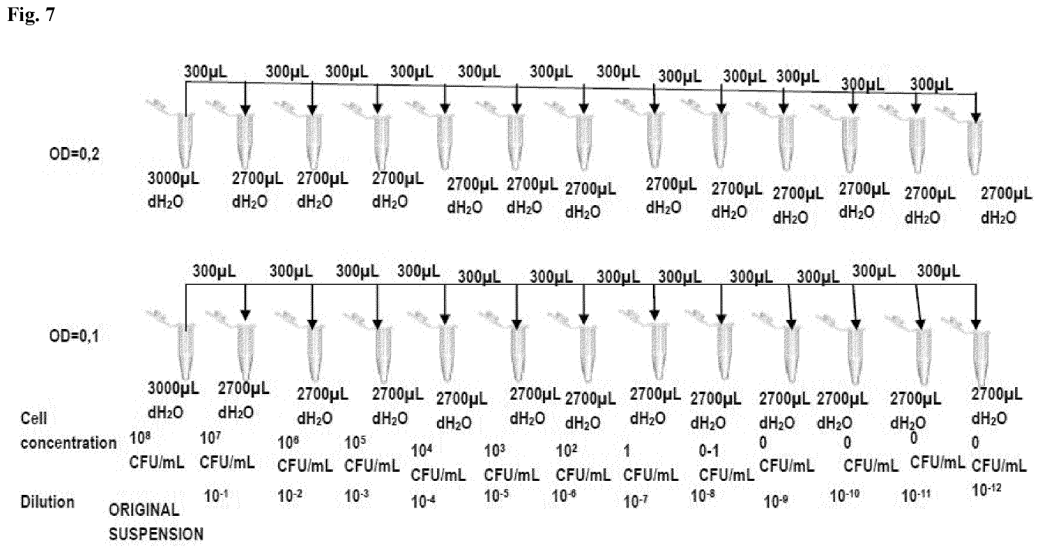

In this Example 3 the correlation curve provided in Example 2 was confirmed. For the proof of concept of the MPN model, an initial test suspension of E. coli was prepared with its concentration calibrated at 10⁸ CFU/mL (Example 4). In Example 3, we checked whether OD 0.1 or 0.2 is a better initial OD to prepare the original bacterial suspension calibrated at 10⁸ CFU/mL, for later experiments (Example 4), thus confirming the linear correlation curve obtained at Example 2. In this Example 3, E. coli was introduced into 3 mL sterile distilled water via inoculation loop upto two different optical densities (OD) 0.1 and 0.2. Afterwards, series of dilutions of 10 were made as in the provided scheme below, transfering 300 μL of the previous dilution into 2700 μL of sterile distilled water as is represented in FIG. 7. At OD 0.2, 1 mL of each of the dilutions 10⁻⁶, 10⁻⁷, 10⁻⁸, 10⁻⁹, 10⁻¹⁰ and 10⁻¹¹ were transfered into 99 mL of sterile distilled water (dH₂O) and filtered through a membrane filter of pore size 0.45 μm. At OD 0.1 1 mL dilutions 10⁻⁷, 10⁻⁸, 10⁻⁹, 10⁻¹⁰, 10⁻¹¹ in 10⁻¹² were transfered into 99 mL of sterile distilled dH2O and filtered through a cellulose filter of pore size 0.45 μm. The dilutions are depicted in FIG. 7.

The results depicted in Table 6 below showed that 0.1 is a better OD of the original suspension in the proof of concept experiments.

TABLE 6

| | 10⁻⁶ | 10⁻⁷ | 10⁻⁸ | 10⁻⁹ | 10⁻¹⁰ | 10⁻¹¹ | 10⁻¹² |
|---|---|---|---|---|---|---|---|
| OD 0.2 | 183 | 20 | 3 | 1 | 0 | 0 | / |
| OD 0.1 | / | 22 | 5 | 1 | 0 | 0 | 0 |

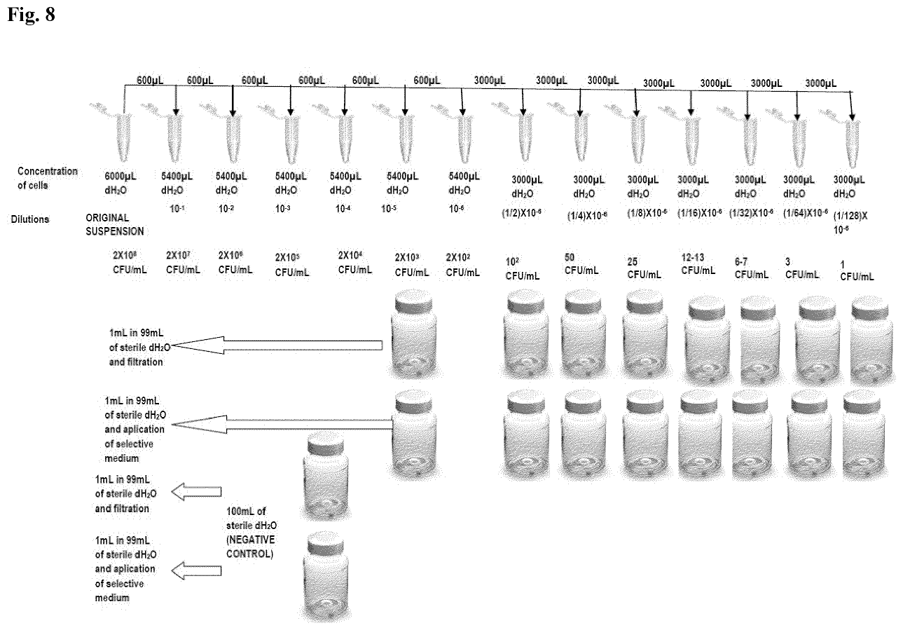

Based on the found optimal OD 0.1 for the calibration of the original test suspension to the concentration 10⁸ CFU/mL the E. coli cells will be introduced into 6 mL of sterile distilled water via an inoculation loop to the final OD of 0.1 generating the original bacterial suspension. From the original bacterial suspension, a dilution series will be made as depicted in FIG. 8 (Example 4) to reach the final predicted concentrations of bacteria 1 CFU/mL, 3 CFU/mL, 6 CFU/mL, 12-13 CFU/mL, 25 CFU/mL, 50 CFU/mL and 100 CFU/mL. 1 mL of dilutions with the predicted concentration of bacteria 1 CFU/mL, 3 CFU/mL, 6 CFU/mL, 12-13 CFU/mL, 25 CFU/mL, 50 CFU/mL and 100 CFU/mL will be transfered to 99 mL of sterile distilled water with solubilized selective, differential chromogenic medium for detection selective for E. coli to reach the final concentrations 1 CFU/100 mL, 3 CFU/100 mL, 6 CFU/100 mL, 12-13 CFU/100 mL, 25 CFU/100 mL, 50 CFU/100 mL and 100 CFU/100 mL. The spiked samples in the selective lyophilized chromogenic medium with the concentrations 1 CFU/100 mL, 3 CFU/100 mL, 6 CFU/100 mL, 12-13 CFU/100 mL, 25 CFU/100 mL, 50 CFU/100 mL and 100 CFU/100 mL will then be transfered to separate MPN sample holders of the present invention and incubated for 24 h at 36° C. A negative control of 1 mL of sterile distilled water will also be added to to 99 mL of sterile distilled water with solubilized selective, differential chromogenic medium for detection selective for E. coli. Simultaneously, 1 mL of dilutions with the predicted concentration of bacteria 1 CFU/mL, 3 CFU/mL, 6 CFU/mL, 12-13 CFU/mL, 25 CFU/mL, 50 CFU/mL and 100 CFU/mL will be transfered to 99 mL of sterile distilled water to reach the final concentrations 1 CFU/100 mL, 3 CFU/100 mL, 6 CFU/100 mL, 12-13 CFU/100 mL, 25 CFU/100 mL, 50 CFU/100 mL and 100 CFU/100 mL. The spiked samples of concentrations 1 CFU/100 mL, 3 CFU/100 mL, 6 CFU/100 mL, 12-13 CFU/100 mL, 25 CFU/100 mL, 50 CFU/100 mL and 100 CFU/100 mL in sterile distilled water will be filtered through a membrane filter of pore size 0.45 μm. A negative control of 1 mL of sterile distilled water will aslo be added to to 99 mL of sterile distilled water and further filtered through a membrane filter of pore sizes 0.45 μm. The filters with the filtered samples will then be transfered to a solid differential chromogenic agar medium, rehydrated with 1 mL of sterile distilled water and the samples on the solid differential chromogenic agar medium will be incubated for 24 h at 36° C.

### Example 4—Proof of Concept of the MPN Model

Based on the found optimal OD 0.1 for the calibration of the original test suspension to the concentration 10⁸ CFU/mL the E. coli cells was introduced into 6 mL of sterile distilled water via an inoculation loop to the final OD of 0.1 generating the original bacterial suspension. From the original bacterial suspension, a dilution series was made as depicted in FIG. 8 (Example 4) to reach the final predicted concentrations of bacteria 1 CFU/mL, 3 CFU/mL, 6 CFU/mL, 12-13 CFU/mL, 25 CFU/mL, 50 CFU/mL and 100 CFU/mL. 1 mL of dilutions with the predicted concentration of bacteria 1 CFU/mL, 3 CFU/mL, 6 CFU/mL, 12-13 CFU/mL, 25 CFU/mL, 50 CFU/mL and 100 CFU/mL were transfered to 99 mL of sterile distilled water with solubilized selective, differential chromogenic medium for detection selective for E. coli to reach the final concentrations 1 CFU/100 mL, 3 CFU/100 mL, 6 CFU/100 mL, 12-13 CFU/100 mL, 25 CFU/100 mL, 50 CFU/100 mL and 100 CFU/100 mL. The spiked samples in the selective lyophilized chromogenic medium with the concentrations 1 CFU/100 mL, 3 CFU/100 mL, 6 CFU/100 mL, 12-13 CFU/100 mL, 25 CFU/100 mL, 50 CFU/100 mL and 100 CFU/100 mL were then transfered to separate MPN sample holders of the present invention and incubated for 24 h at 36° C. A negative control of 1 mL of sterile distilled water was also added to to 99 mL of sterile distilled water with solubilized selective, differential chromogenic medium for detection selective for E. coli. Simultaneously, 1 mL of dilutions with the predicted concentration of bacteria 1 CFU/mL, 3 CFU/mL, 6 CFU/mL, 12-13 CFU/mL, 25 CFU/mL, 50 CFU/mL and 100 CFU/mL were transfered to 99 mL of sterile distilled water to reach the final concentrations 1 CFU/100 mL, 3 CFU/100 mL, 6 CFU/100 mL, 12-13 CFU/100 mL, 25 CFU/100 mL, 50 CFU/100 mL and 100 CFU/100 mL. The spiked samples of concentrations 1 CFU/100 mL, 3 CFU/100 mL, 6 CFU/100 mL, 12-13 CFU/100 mL, 25 CFU/100 mL, 50 CFU/100 mL and 100 CFU/100 mL in sterile distilled water were filtered through a membrane filter of pore size 0.45 μm. A negative control of 1 mL of sterile distilled water was also added to 99 mL of sterile distilled water and further filtered through a membrane filter of pore sizes 0.45 μm. The filters with the filtered samples were then transfered to a solid differential chromogenic agar medium, rehydrated with 1 mL of sterile distilled water and the samples on the solid differential chromogenic agar medium were incubated for 24 h at 36° C. Results are depicted in the Table 7 below.

TABLE 7

| | | Lower limit of number of | Upper limit of number of | |
|---|---|---|---|---|
| | | cells in 100 mL of water | cells in 100 mL of water | |
| | | sample based on number of | sample based on number of | |
| | | positive compartments | positive compartments | Number of cells in |
| | | out of all three | over all three | 100 mL of water sample |
| | | compartments of equal | compartments of equal | based on the alternative |
| | | volume and over all | volume and over all | method of membrane filtration |
| | | eight different volumes | eight different volumes | (ISO 9308-1: 2014; Water |
| | | and based on the | and based on the | quality - Enumeration of |
| Predicted | | statistical calculation | statistical calculation | Escherichia coli |
| concentrational | | of lower limits of | of upper limits of | and coliform bacteria - |
| range of cell | Value of MPN | 95% confidence | 95% confidence | Part 1: Membrane filtration |
| culture | estimate | intervals | intervals | method for waters with low |
| (CFU/100 mL) | (CFU/100 mL) | (CFU/100 mL) | (CFU/100 mL) | bacterial background flora) |
| 0 | 0 | 0 | 0 | 0 |
| 1 | 1 | 1 | 8 | 1 |
| 3 | 3 | 3 | 10 | 2 |
| 6 | 6 | 5 | 14 | 5 |
| 12-13 | 13 | 10 | 25 | 14 |
| 25 | 19 | 12 | 35 | 28 |
| 50 | 54 | 32 | 93 | 56 |
| 100 | 93 | 47 | 190 | 114 |

Apparently there is a good coherence between the predicted concentration of E. coli, the MPN estimate at the predicted concentration and the result of the membrane filtration. At the pedicted concentration of 1 CFU/mL, the MPN sample holder predicted the most probable number of bacterial cells in 100 mL of the sample to be 1 CFU. The statistical analysis in combintation with the actual total number of positive wells predicted the actual number of bacteria in the 100 mL of water sample to range between 1 CFU and 8 CFU and the membrane filtration resulted in 1 CFU/100 mL of sample. At the pedicted concentration of 3 CFU/mL, the MPN sample holder predicted the most probable number of bacterial cells in 100 mL of the sample to be 3 CFU. The statistical analysis in combination with the actual total number of positive wells predicted the actual number of bacteria in the 100 mL of water sample to range between 3 CFU and 10 CFU and the membrane filtration resulted in 2 CFU/100 mL of sample. At the pedicted concentration of 6 CFU/mL, the MPN sample holder predicted the most probable number of bacterial cells in 100 mL of the sample to be 6 CFU. The statistical analysis in combination with the actual total number of positive wells predicted the actual number of bacteria in the 100 mL of water sample to range between 5 CFU and 14 CFU and the membrane filtration resulted in 5 CFU/100 mL of sample. At the pedicted concentration of 12-13 CFU/mL, the MPN sample holder predicted the most probable number of bacterial cells in 100 mL of the sample to be 13 CFU. The statistical analysis in combintation with the actual total number of positive wells predicted the actual number of bacteria in the 100 mL of water sample to range between 10 CFU and 25 CFU and the membrane filtration resulted in 14 CFU/100 mL of sample. At the pedicted concentration of 25 CFU/mL, the MPN sample holder predicted the most probable number of bacterial cells in 100 mL of the sample to be 19 CFU. The statistical analysis in combination with the actual total number of positive wells predicted the actual number of bacteria in the 100 mL of water sample to range between 12 CFU and 35 CFU and the membrane filtration resulted in 28 CFU/100 mL of sample. At the pedicted concentration of 50 CFU/mL, the MPN sample holder predicted the most probable number of bacterial cells in 100 mL of the sample to be 54 CFU. The statistical analysis in combination with the actual total number of positive wells predicted the actual number of bacteria in the 100 mL of water sample to range between 32 CFU and 93 CFU and the membrane filtration resulted in 56 CFU/100 mL of sample. At the pedicted concentration of 100 CFU/mL, the MPN sample holder predicted the most probable number of bacterial cells in 100 mL of the sample to be 93 CFU. The statistical analysis in combination with the actual total number of positive wells predicted the actual number of bacteria in the 100 mL of water sample to range between 47 CFU and 190 CFU and the membrane filtration resulted in 114 CFU/100 mL of sample. If a defined number of compartments are positive out of all compartments, this may indicate the minimum number of cells present in the sample as each positive compartment surely contains a minimum of one cell. Each compartment may however in coherence with the Poisson distribution may contain more than one cell which may, depending on the volume of the compartment be more probable (according to the Poisson distribution) than containig only one cell.

1. A method for detection and/or quantification of microorganism in a liquid sample, in particular in a water sample, the method comprising the steps of:
(a-1) distributing the liquid sample into a number of different discrete volume portions, wherein the different discrete volume portions define linearly increasing volumes, or
(a-2) diluting the liquid sample into a number of dilution samples which are defined by linearly increasing dilutions;
(b) allowing the microorganism to grow; and
(c) applying the Most Probable Number method to the linearly distributed volume portions or the linearly diluted dilution samples to detect and/or quantify the microorganism.

2. (canceled)

3. The method according to claim 1, wherein the linearly increasing volumes represent linearly increasing discrete volume portions—with different volumes following the linearly increasing distribution of the linear set:
1x-, 2x-, 3x-, 4x- and 5x-fold increasing volumes;
1x-, 2x-, 3x-, 4x-, 5x-, 6x-, 7x- and 8x-fold increasing volumes;
1x-, 2x-, 3x-, 4x-, 5x-, 6x-, 7x-, 8x- and 9x-fold increasing volumes;
and subsequent linearly increasing volume distributions correspondingly increased by a further number, up to 1x-, 2x-, 3x-, 4x-, 5x-, 6x-, 7x-, 8x-, 9x-, 10x-, 11x-, 12x-, 13x-, 14x-, 15x-, 16x-, 17x-, 18x-, 19x-and 20x-fold increasing volumes.

4. The method according to claim 1, wherein the linearly increasing volumes—or the linearly decreasing dilutions comprises at least triplicate repetitions per each distinct volume portion in the linear distribution of the different discrete volume portions.

5. The method according to claim 1, wherein the liquid sample subjected to step (a) has a volume of about 100 mL, and/or wherein the liquid sample subjected to step (a) contains less than 100 CFU microorganisms per 100 mL.

6. The method according to claim 1, wherein the liquid sample is selected from the group consisting of:
drinking water
surface or natural water,
bathing water,
industrial process water,
wastewater, and
recycled wastewater.

7. The method according to claim 1, wherein the liquid sample is obtained from wastewater, industrial processing water, natural water, bathing water, industrial process water and/or recycled wastewater, wherein said liquid sample is diluted once before subjecting said water sample to step (a).

8. The method according to claim 1, which method further comprises, prior to step (a), suspending a defined sterile lyophilized medium with microorganism-specific detection reagents in a fixed amount of the liquid sample, then distributing said liquid sample in step (a) on a sample holder.

9. The method according to claim 1, wherein the lower limits of 95% confidence intervals linearly increase from 0.001 CFU/mL at MPN estimate of 1 CFU/100 mL to 0.04 CFU/mL at MPN estimate 10 CFU/100 mL and to 0.2 CFU/mL at MPN estimate 40 CFU/mL, and the upper limits of the 95% confidence intervals linearly increase from 0.07 CFU/mL at MPN estimate of 1 CFU/100 mL to 0.2 CFU/mL at MPN estimate 10 CFU/100 mL and to 0.7 CFU/mL at MPN estimate 40 CFU/mL.

10. The method according to claim 1, wherein the presence of bacteria is detected automatically by a positive signal in a defined volume, out of all repetitions of the defined volume and over all different volumes.

11. A sample holder for detection and/or quantification of microorganism in a liquid sample,
wherein the sample holder is structured to hold the liquid sample in a number of different compartments, wherein the different compartments respectively define linearly increasing volumes, wherein a compartment defining each of the respective linearly increasing volume is present in triplicate of a same volume each, thereby optionally forming in total 15 compartments in case of 1x-, 2x-, 3x-, 4x- and 5x-fold linear increasing volumes; forming in total 24 compartments in case of 1x-, 2x-, 3x-, 4x-, 5x-, 6x-, 7x- and 8x-fold linear increasing volumes; up to forming 60 compartments in case of 1x-, 2x-, 3x-, 4x-, 5x-, 6x-, 7x-, 8x-, 9x-, 10x-, 11x-, 12x-, 13x-, 14x-, 15x-, 16x-, 17x-, 18x-, 19x-and 20x-fold linear increasing volumes.

12. The sample holder according to claim 11, wherein the different compartments are structured or sized to respectively define a
linear distribution consisting of 1x-, 2x-, 3x-, 4x-, 5x-, 6x-, 7x- and 8x-fold linear increasing volumes.

13. (canceled)

14. The sample holder according to claim 11, wherein the sample holder has an outer shape selected from a cylindrical outer shape, a spherical outer shape, a rectangular outer shape or other shape, and/or wherein the sample holder has varying dimensions in terms of height, width and/or radius.

15. The sample holder according to claim 11, wherein the compartments are organized in a spiral order with a defined distance between the compartments and a defined curvature.

16. The sample holder according to claim 11, wherein when the total volume to hold the sample is given as V=100%, for each of three compartments of the linear volume distribution the 1x unit volume is 0.926% of V, the 2x-fold unit volume is 1.852% of V, the 3x-fold unit volume is 2.778% of V, the 4x-fold unit volume is 3.704% of V, the 5x-fold unit volume is 4.63% of V, the 6x-fold unit volume is 5.556% of V, the 7x-fold unit volume is 6.481% of V, and the 8x-fold unit volume is 7.407% of V, wherein each %-volume indication encompasses a ±10% volume tolerance range, and optionally wherein V=100% is 100 mL.

17. The sample holder according to claim 11, having a circular outer shape whose compartments divide the circular outer shaped sample holder into radial sections to define said number of compartments respectively defining the linear volume distribution; or having a rectangular outer shape whose compartments divide the sample holder into rectangular sections having said number of compartments respectively defining the linear volume distribution.

18. (canceled)

19. The sample holder according to claim 11, wherein the holder-forming material is made of a hydrophobic material, or is provided with a hydrophobic coating of at least at a part of or all of the surface facing the holder inner space.

20. The sample holder according claim 11, which sample holder further comprises a cover, wherein the cover is arranged for preventing fluid evaporation, and/or is arranged preventing fluid exchange between compartments of the sample holder.

21. (canceled)

22. A system comprising:
a sample holder as defined in claim 11, and a detector for detecting the presence of bacteria by a positive signal in a defined volume, out of all repetitions of the defined volume and over all different volumes, in an automatized form.

23. The method according to claim 1, wherein the linearly increasing distribution consists of 1x-, 2x-, 3x-, 4x-, 5x-, 6x-, 7x- and 8x-fold increasing volumes.

24. The system according to claim 19, wherein the system is adapted to automatically calculate the Most Probable Number from the positive signal results, and optionally the system is further adapted to automatically generate or calculate the upper and lower limits of the 95% confidence intervals.
